(19) United States
(12) Patent Application Publication
(10) Pub. No.: US 2004/0140215 A1
(43) Pub. Date: Jul. 22, 2004

(54) Pre-cast electrophoresis slab gels with extended storage life

(75) Inventor: Cory M. Panattoni, Winters, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: BIO-RAD LABORATORIES, INC., Hercules, CA

(21) Appl. No.: 10/346,681

(22) Filed: Jan. 17, 2003

(51) Int. Cl. G01L 1/20; B01D 57/02; C07K 1/26

(52) U.S. Cl. 204/466; 204/467; 204/617; 204/618; 204/620; 204/621

(57) ABSTRACT

In pre-cast slab gel cassettes, the formation of pathways in which proteins can migrate between the gel and the walls of the cassette to form shadow bands is avoided by including a nonionic amphiphilic polymer in the monomer solution from which the gel is formed and casting the gel with the polymer included. The nonionic amphiphilic polymer also prevents the resulting gel from sticking to the walls when the gel is to be removed from the cassette after electrophoresis.

### BACKGROUND OF THE INVENTION

This invention relates to polyacrylamide gels as used in slab gel electrophoresis.

When electrophoresis is performed in a slab gel, several samples can be analyzed simultaneously in the same gel and the resulting electropherograms can be observed and read visually by identifying the locations of the bands on the gel that correspond to the individual components. Polyacrylamide is a gel material that is widely used in slab gels.

Slab gels are frequently supplied in pre-cast form in cassettes that typically contain two flat transparent plates with the gel retained between them. The plates may be glass or plastic, one commonly used plastic being a polystyrene-acrylonitrile blend. A difficulty with certain pre-cast polyacrylamide gels is that during storage the gels appear to separate from the cassette plates. This creates a pathway between the gel and one or both of the plates in which the sample can migrate during electrophoresis. This migration causes shadow bands in the electropherogram which obscure the clarity and identification of the parent bands, i.e., those that are formed as a direct result of the electrophoretic separation. Shadow bands occur most frequently in pre-cast gels that have been stored without cooling.

Another problem encountered with polyacrylamide slab gels is a tendency of the gels to stick or adhere to the plates. This presents a difficulty once the separation is completed and the gel must be removed from the plates for purposes of staining, photographing or other observation, detection or recordation. Attempts to remove a gel that is sticking to one or both of the plates can result in a damaged gel and a ruined experiment. This problem is especially acute for gels of low concentration and for gels used for isoelectric focusing.

The polymerization reaction to form polyacrylamide is inhibited when dissolved oxygen is present in the gel-forming liquid at or near the gel plate. This is especially true when the gel plates are plastic, such as polystyrene-acrylonitrile, for example. To prevent this inhibition from occurring, a coating of polyvinylidene chloride or polyvinyl dichloride (PVDC) is often applied to the plates prior to contacting the plates with the polyacrylamide gel material. Unfortunately, these coatings produce an effect on the electrophoresis image that appears to be the result of separation between the gel and the plate. These coatings also exacerbate the sticking problem when the gel is an isoelectric focusing gel, for example one with a pH ranging from 5 to 8.

### SUMMARY OF THE INVENTION

The present invention resides in the discovery that both the occurrence of shadow bands due to apparent pathways between a polyacrylamide gel and a gel cassette plate and the adherence of the gel to the plate can be prevented by forming the gel from a monomer solution that includes a nonionic amphiphilic polymer in addition to the monomers. The polymer is added to the solution before the gel is cast, and casting is then performed with the polymer still present.

### DETAILED DESCRIPTION OF THE INVENTION AND PREFERRED EMBODIMENTS

Examples of nonionic amphiphilic polymers that can be used in the practice of this invention are polyvinyl alcohol, agarose, polyvinyl pyrrolidone, polyethylene glycol, polypropylene glycol, polypropylene glycol/polyethylene glycol copolymers, and linear polyacrylamide. These polymers are fully formed prior to being added to the gel-forming solution, are soluble in the gel-forming solution, and do not have sites available for crosslinking reactions. Preferred polymers are those having molecular weights of about 100,000 or less, more preferred are those with molecular weights of about 20,000 or less, still more preferred are those with molecular weights within the range of about 200 to about 20,000, and still more preferred are those with molecular weights within the range of about 200 to about 5,000. The weight percent of the polymer in the monomer solution can range widely, although lowering the molecular weight tends to permit equivalent or similar results with higher weight percents of the polymer. In the case of polyvinyl alcohol, for example, a preferred concentration range is from about 0.5% to about 5% by weight of the monomer solution. When polyethylene glycol is used, a preferred concentration is from about 0.01% to about 0.3% by weight. The concentrations and molecular weights of other nonionic amphiphilic polymers are readily determined by routine experimentation and will in many cases be readily apparent to those skilled in the art.

The gel-forming solution is an aqueous solution of a monomer mixture that is polymerizable, generally by a free-radical reaction, to form polyacrylamide. Any monomer mixture that has been used or is described in the literature as being useful in forming polyacrylamide gels can be used in the practice of this invention. The monomer mixture typically includes acrylamide, a crosslinking agent, and a free radical initiator. Preferred crosslinking agents are bisacrylamides, and a particularly convenient crosslinking agent is N,N′-methylene-bisacrylamide.

The gel-forming solution will also typically include a free radical initiator system. The most common system used is N,N,N′,N′-tetramethylenediamine (TEMED) in combination with ammonium persulfate. Other systems will be apparent to those skilled in the art. The gel-forming solution can also contain additional components that are known or used in electrophoresis gels for various reasons. Buffering agents are commonly included since electrophoretic separations are typically performed at designated pH values. Density control agents, such as glycerol, are also useful in many systems, particularly when the resolving gel is formed underneath a stacking gel.

Among those skilled in the use of electrophoresis and the preparation of electrophoresis gels, polyacrylamide gels are characterized by the parameters T and C, which are expressed as percents and defined as follows (in which “bis” denotes the bisacrylamide crosslinker):

$$\begin{matrix}T = \frac{( combined weight of acrylamide and bis in grams )}{( volume of aqueous solution in mL )} \times 100 \\ C = \frac{( weight of bis )}{( combined weight of acrylamide and bis )} \times 100\end{matrix}$$

The values of T and C can vary in the present invention as they do in the use of polyacrylamide gels in general. For the purposes of the present invention, a preferred range of T values is from about 3% to about 30%, and most preferably from about 5% to about 20%. A preferred range of C values of from about 1% to about 10% (corresponding to a range of weight ratio of acrylamide to bisacrylamide of from about 10:1 to about 100:1), and most preferably from about 2% to about 4% (corresponding to a range of weight ratio of acrylamide to bisacrylamide of from about 25:1 to about 50:1).

The invention is applicable to gels of uniform concentration as well as gradient gels. The methods for forming both uniform and gradient gels are well known in the art.

The plates that form the gel cassette are chemically inert, transparent materials, either glass or plastic or both. A wide variety of plastics can be used. The plastics are generally injection moldable plastics, and the selection is limited only by the need for the plastic to be inert to the gel-forming solution, the gel itself, the solutes (typically proteins) in the samples to be analyzed in the cassette, the buffering agents, and any other components that are typically present in the samples. Examples of these plastics are polycarbonate, polystyrene, acrylic polymers, styrene-acrylonitrile copolymer (SAN, NAS), BAREX® acrylonitrile polymers (Barex Resins, Naperville, Ill., USA), polyethylene terephthalate (PET), polyethylene terephthalate glycolate (PETG), and poly(ethylene naphthalenedicarboxylate) (PEN).

The following example is offered for illustrative purposes and are not intended to limit the scope of the invention.

### EXAMPLE

Three aqueous gel-forming solutions to be used in the formation of a gradient gel were prepared as follows (all percents by weight):

Solution A:

acrylamide/N,N′-methylene-bisacrylamide (T=21%, C=2.6%)

10% glycerol

0.1% TEMED

0.0375% polyethylene glycol, weight-average molecular weight 200-1,000

Solution B:

acrylamide/N,N′-methylene-bisacrylamide (T=6%, C=2.6%)

0.2% TEMED

0.0375% polyethylene glycol, weight-average molecular weight 200-1,000

Solution C:

1.125 M tris-HCl (tris(hydroxymethyl)aminomethane hydrochloride), pH 8.6

0.15% ammonium persulfate

A slab gel cassette formed from two styrene-acrylonitrile plastic plates was used, with a gel space measuring 13.4 cm×8.4 cm×1 mm. A gel was formed inside the cassette by first pumping a mixture of Solution B and Solution C at a volume ratio of two-thirds B to one-third C into the cassette from the bottom, to achieve a T=4% stacking gel solution with a PEG concentration of 0.025% by weight. A gradient gel was then formed under the stacking gel by pumping a mixture of Solutions A, B, and C at varying amounts of A and B into the cassette under the 4% gel solution. A ratio of two parts by volume of A plus B to one part by volume of C was maintained while the volume ratio of A to B was varied to produce a T gradient extending from 10.5% to 14%.

The foregoing description is primarily for purposes of illustration. Further modifications, substitutions and variations will be apparent to those skilled in the art and will be included within the scope of the invention.

What is claimed is:

1. A method for manufacturing a pre-cast polyacrylamide slab gel for use in slab electrophoresis, said method comprising:
(a) placing a gel-forming liquid mixture inside a gel enclosure defined by a pair of chemically inert, transparent plates separated from each other by fixed distance, said gel-forming mixture comprising an acrylamide monomer, a crosslinking agent, a buffer, and a nonionic amphiphilic polymer having a molecular weight of about 100,000 or less, in aqueous solution; and
(b) polymerizing said gel-forming mixture into a gel.

2. A method in accordance with claim 1 in which said nonionic amphiphilic polymer has a molecular weight of about 20,000 or less.

3. A method in accordance with claim 1 in which said nonionic amphiphilic polymer is a member selected from the group consisting of polyvinyl alcohol, agarose, polyvinyl pyrrolidone, polyethylene glycol, polypropylene glycol, polypropylene glycol/polyethylene glycol copolymers, and linear polyacrylamide.

4. A method in accordance with claim 1 in which said nonionic amphiphilic polymer is polyvinyl alcohol.

5. A method in accordance with claim 4 in which said polyvinyl alcohol has a molecular weight of from about 200 to about 20,000.

6. A method in accordance with claim 4 in which said polyvinyl alcohol comprises from about 0.5% to about 5% by weight of said aqueous solution.

7. A method in accordance with claim 1 in which said nonionic amphiphilic polymer is polyethylene glycol.

8. A method in accordance with claim 7 in which said polyethylene glycol has a molecular weight of from about 200 to about 20,000.

9. A method in accordance with claim 7 in which said polyethylene glycol comprises from about 0.01% to about 0.3% by weight of said aqueous solution.

10. A method in accordance with claim 1 in which said plates are glass.

11. A method in accordance with claim 1 in which said plates are plastic.

12. A method in accordance with claim 11 in which said plastic is a member selected from the group consisting of polycarbonate, polystyrene, acrylic polymers, styrene-acrylonitrile copolymer, acrylonitrile polymers, polyethylene terephthalate, polyethylene terephthalate glycolate, and poly(ethylene naphthalenedicarboxylate).

13. A method in accordance with claim 11 in which said plastic is a polystyrene-acrylonitrile blend.

14. A pre-cast polyacrylamide slab gel for use in slab gel electrophoresis, said pre-cast slab gel comprising:
a pair of chemically inert, transparent plates, and
a polyacrylamide gel cast between said plates, said polyacrylamide gel formed by polymerization of an acrylamide monomer and a crosslinking agent, said polymerization having been performed in an aqueous solution comprising said acrylamide monomer, said crosslinking agent, a buffer, and a nonionic amphiphilic polymer having a molecular weight of about 100,000 or less.

15. A pre-cast polyacrylamide slab gel in accordance with claim 14 in which said nonionic amphiphilic polymer has a molecular weight of about 20,000 or less.

16. A pre-cast polyacrylamide slab gel in accordance with claim 14 in which said nonionic amphiphilic polymer is a member selected from the group consisting of polyvinyl alcohol, agarose, polyvinyl pyrrolidone, polyethylene glycol, polypropylene glycol, polypropylene glycol/polyethylene glycol copolymers, and linear polyacrylamide.

17. A pre-cast polyacrylamide slab gel in accordance with claim 14 in which said nonionic amphiphilic polymer is polyvinyl alcohol.

18. A pre-cast polyacrylamide slab gel in accordance with claim 17 in which polyvinyl alcohol has a molecular weight of from about 200 to about 20,000.

19. A pre-cast polyacrylamide slab gel in accordance with claim 17 in which said polyvinyl alcohol comprises from about 0.5% to about 5% by weight of said aqueous solution.

20. A pre-cast polyacrylamide slab gel in accordance with claim 14 in which said nonionic amphiphilic polymer is polyethylene glycol.

21. A pre-cast polyacrylamide slab gel in accordance with claim 20 in which said polyethylene glycol has a molecular weight of from about 200 to about 20,000.

22. A pre-cast polyacrylamide slab gel in accordance with claim 20 in which said polyethylene glycol comprises from about 0.01% to about 0.3% by weight of said aqueous solution.

23. A pre-cast polyacrylamide slab gel in accordance with claim 14 in which said plates are glass.

24. A pre-cast polyacrylamide slab gel in accordance with claim 14 in which said plates are plastic.

25. A pre-cast polyacrylamide slab gel in accordance with claim 24 in which said plastic is a member selected from the group consisting of polycarbonate, polystyrene, acrylic polymers, styrene-acrylonitrile copolymer, acrylonitrile polymers, polyethylene terephthalate, polyethylene terephthalate glycolate, and poly(ethylene naphthalenedicarboxylate).

26. A pre-cast polyacrylamide slab gel in accordance with claim 24 in which said plastic is a polystyrene-acrylonitrile blend.
